(19) United States
(12) Patent Application Publication
(10) Pub. No.: US 2009/0270657 A1
(43) Pub. Date: Oct. 29, 2009

(54) PROCESS FOR THE PREPARATION OF A 1,2-ALKYLENE DIOL AND A DIALKYLCARBONATE

(76) Inventors: Evert Van Der Heide, Amsterdam (NL); Cornelis Leonardus Maria Vrouwenvelder, Amsterdam (NL)

Correspondence Address: SHELL OIL COMPANY, P O BOX 2463, HOUSTON, TX, 772522463, US

(21) Appl. No.: 12/108,299

(22) Filed: Apr. 23, 2008

(30) Foreign Application Priority Data
Apr. 23, 2008 (EP) 07106705.2

(51) Int. Cl. C07C 31/18

(52) U.S. Cl. 568/852

(57) ABSTRACT

The present invention relates to a process for the preparation of a 1,2-alkylene diol and a dialkylcarbonate, comprising the steps of
(i) contacting a 1,2-alkylene oxide with carbon dioxide in the presence of a carbonation catalyst in a downflow jet reactor to obtain a reaction mixture containing a 1,2-alkylene carbonate, wherein the downflow jet reactor further comprises a deflection means situated in between the ejector means and the outlet means in the direction of the flow path of the gas/liquid mixture generated by the ejector means;
(ii) contacting at least part of the reaction mixture obtained in step (i) with an alkanol to obtain a reaction mixture containing a 1,2-alkylene diol and a dialkylcarbonate; and
(iii) recovering the 1,2-alkylene diol and the dialkylcarbonate from the reaction mixture obtained in step (ii).

### CROSS REFERENCE TO RELATED APPLICATION

This application claims priority to European Patent Application number EP 07106705.2 filed Apr. 23, 2007, the entire disclosure of which is herein incorporated by reference.

### FIELD OF THE INVENTION

The present invention relates to a process for the preparation of a 1,2-alkylene diol and a dialkylcarbonate, from carbon dioxide, a 1,2-alkylene oxide and an alkanol.

### BACKGROUND OF THE INVENTION

1,2-Alkylene diols (commonly referred to as mono-alkylene glycol), such as 1,2-ethylene diol (ethylene glycol) and 1,2-propylene diol (propylene glycol), are useful as specialty solvents and intermediates in numerous chemical processes. Furthermore, 1,2-propylene diol also has other uses such as additives for pharmaceutical drugs and food due to its low toxicity for human beings.

In addition dialkylcarbonates can be a useful intermediate in the preparation of diphenylcarbonate, which is an important polymer intermediate.

Such 1,2-alkylene diols and dialkylcarbonates may be prepared via an intermediate 1,2-alkylene carbonate.

Such a process is described in Chinese patent application CN 1528735. This document describes a process in which carbon dioxide is reacted with an alkylene oxide to yield alkylene carbonate, e.g. propylene carbonate or ethylene carbonate. The alkylene carbonate is subjected to transesterification using an alkanol, e.g. methanol, in a reactive distillation column to prepare alkylene glycol and dialkylcarbonate.

Processes for the production of alkylene carbonates have been described in the prior art. For example WO 2005/003113 discloses a process in which carbon dioxide is contacted with an alkylene oxide in the presence of a suitable catalyst. The catalyst is recycled to the alkylene carbonate preparation in an alcohol, in particular in propylene glycol (1,2-propane diol).

U.S. Pat. No. 6,080,897 describes a method for producing monoethylene glycol, which comprises a carbonation step in which ethylene oxide is allowed to react with carbon dioxide in the presence of a carbonation catalyst to form a reaction solution containing ethylene carbonate, a hydrolysis step in which the reaction solution is converted with water into an ethylene glycol aqueous solution and a distillation step in which purified ethylene glycol and a catalyst solution containing ethylene glycol are obtained. The carbonation reaction is carried out in the presence of a carbonation catalyst using a bubble column reactor. As shown in the examples the bubble column reactor is an upflow reactor. Reactants are fed into the reactor from the bottom, or via a sparger located in the lower half of the reactor. A part of the reaction solution is recycled from the top of the reactor to the bottom of the reactor.

Korean application KR 20060130395 describes a process for producing ethylene carbonate by reacting ethylene oxide with carbon dioxide in a loop reactor equipped with an ejector. According to the application, the reactants are fed into the reactor from the top. A part of the reaction solution which flows downwardly through the reactor, is recycled from the bottom of the reactor to the top of the reactor. In the upper part of the reactor, an ejector means is situated which effects mixing of the carbon dioxide and the recycled reaction liquid. The application does not disclose reacting a 1,2-alkylene oxide with carbon dioxide into a 1,2-alkylene carbonate, and subsequently reacting the 1,2-alkylene carbonate thus obtained with an alkanol to produce a 1,2-alkylene diol and a dialkylcarbonate.

It would be desirable to make more efficient use of the carbon dioxide gaseous reactant, and to prevent unreacted carbon dioxide from leaving the reactor as much as possible.

### SUMMARY OF THE INVENTION

It has now been found that the above-mentioned desire is satisfied by a process for the preparation of a 1,2-alkylene diol and a dialkylcarbonate, comprising the steps of
(i) contacting a 1,2-alkylene oxide with carbon dioxide in the presence of a carbonation catalyst in a downflow jet reactor to obtain a reaction mixture containing a 1,2-alkylene carbonate,
wherein the downflow jet reactor is a reactor comprising a reactor vessel, an ejector means suitable for mixing the gas and the liquid and ejecting the gas/liquid mixture obtained into the reactor vessel, and an outlet means, wherein the ejector means is situated in the upper part of the reactor vessel and the outlet means is situated in the lower part of the reactor vessel, which reactor is operated in a downflow fashion, and
wherein the downflow jet reactor further comprises a deflection means situated in between the ejector means and the outlet means in the direction of the flow path of the gas/liquid mixture generated by the ejector means;
(ii) contacting at least part of the reaction mixture obtained in step (i) with an alkanol to obtain a reaction mixture containing a 1,2-alkylene diol and a dialkylcarbonate; and
(iii) recovering the 1,2-alkylene diol and the dialkylcarbonate from the reaction mixture obtained in step (ii).

In accordance with the present invention, the mixture of carbon dioxide gas and liquid 1,2-alkylene oxide and 1,2-alkylene carbonate is deflected on the surface of the deflection means. As a result, the liquid reaction medium is slowed down, and the gas bubbles present in the liquid medium are deflected away from the outlet, thereby advantageously preventing them from leaving the reactor without having reacted.

Further, by using a downflow jet reactor in step (i) of the process of the present invention, there is a thorough mixing of the reactants, an optimal gas/liquid distribution (because of relatively small bubbles a large interfacial area for mass transfer is available), an efficient internal heat transfer, and only a small dead volume or gas cap.

### BRIEF DESCRIPTION OF THE DRAWING

FIG. 1 depicts a preferred embodiment of the downflow jet reactor for contacting gas and liquid that may be used in step (i) of the process of the present invention.

### DETAILED DESCRIPTION OF THE INVENTION

A downflow jet reactor is defined as a reactor comprising: a reactor vessel (a); an ejector means (b) suitable for mixing the gas and the liquid and ejecting the gas/liquid mixture obtained into the reactor vessel (a); and an outlet means (c), wherein the ejector means (b) is situated in the upper part of the reactor vessel (a) and the outlet means (c) is situated in the lower part of the reactor vessel (a), which reactor is operated in a downflow fashion.

In the subject process, carbon dioxide and 1,2-alkylene oxide are fed to the upper part of the reactor and injected downwards into the reaction medium trough an ejector means. The upper part of the reactor is defined as the upper half of the reactor if the reactor is fully filled with one phase or, if the reactor contains two phases (e.g. a gas and a liquid phase), above or in the upper half of the lower (e.g. liquid) phase. Preferably the carbon dioxide and 1,2-alkylene oxide are fed at the top of the reactor.

The downflow jet reactor is equipped with an ejector means for mixing of the gaseous and liquid compounds. The use of such a type of reactor in the highly exothermic carbonation reaction has the advantage that a higher dosage of alkylene oxides can be applied due to the high mass transfer and a better heat dissipation than even with a cascade of several conventional bubble cell reactors can be achieved. This allows for a single downflow jet reactor to replace a cascade of bubble-flow cell reactors in order to achieve a sufficiently high conversion of the alkylene oxide, and hence requires a lower capital expenditure and space. Moreover, in the downflow jet reactor, any gas headspace is preferably continuously recycled to the reactor, thereby eliminating accumulation of alkylene oxide therein, and hence decreased explosion risk.

The downflow jet reactor may be any reactor known to the skilled person to be suitable for this purpose. Suitable downflow jet reactors are for example described in Ullmann's Encyclopedia of Industrial Chemistry, Vol. B4, 1992, page 297-299.

The reactor vessel (a) may be any vessel suitable as a reactor for contacting gas and liquid. It may be a tank reactor, an open-ended conduit, or a tubular reactor, and may be of conical shape, frustroconical shape, cylindrical shape, or any combination of such shapes. The aspect ratio between length and diameter (1/d_{R}) of the reactor may vary. Preferably, the aspect ratio (1/d_{R}) is in the range of from 1 to 60, more preferably from 2 to 35, yet more preferably, the aspect ratio is in the range of from 3 to 10.

The reactor vessel may further comprise an external and/or internal heat exchange system. The reactor vessel further may comprise at least one inlet for gas or liquid additional to the ejector inlet. The reactor vessel (a) is equipped with at least one ejector means (b) for mixing of the gaseous and liquid compounds and for circulating the formed gas/liquid mixture through the reactor. The ejector means (b) may for example be executed as a venturi plate positioned within an open-ended conduit that discharges the gas/liquid mixture into the reactor vessel, or as another example, be positioned within the headspace of the reactor vessel. Hereby, the headspace itself will become the mixing chamber. The ejector means (b) may also be executed as a “gas assist” nozzle where gas expansion is used to drive the nozzle, or as two phase injector jet nozzle, ejector jet, venturi jet or slit jet, preferably with a momentum transfer tube, as described by Wolf-Dieter Deckwer in “Bubble Column Reactors”, Wiley and Sons, 1985, p. 12 and as described in Ullmann's Encyclopedia of Industrial Chemistry, Vol. B4, 1992, p. 297-299, or as two or more ejectors that direct their gas-liquid streams towards each other to impinge, thereby forming a single gas-liquid stream. This has the advantage that the entire gas-phase is continuously circulated into the reaction medium, and no build-up of compounds increasing explosion risks occurs. Most preferred are ejector jet nozzles or ejectors.

Ejector jet nozzles or ejectors are composed of three basic parts: a converging-diverging primary nozzle, a mixing chamber in contact with the headspace of the reactor and a diffuser. A high pressure motivating fluid enters at the converging-diverging primary nozzle. The motivating liquid is then compressed through the primary injection nozzle into a mixing chamber. This creates a pressure differential in the mixing chamber, by which gas or gas-liquid-mixture, also known as suction liquid or suction gas that is present in the headspace of the reactor is drawn into the mixing chamber. Herein, the suction fluid or gas is mixed with the motivating fluid. The gas and liquid phase form a gas-liquid mixture, which is then recompressed through the diffuser and injected into the reactor. The combined components then form a gas-liquid mixture, which is injected into the reactor through the diffuser. In this way, an intimate mixing of the components is achieved. The flow of the motivating fluid through the injection nozzle induces strong gas suction in the headspace of the reactor. In this way, the gas in the headspace including the carbon dioxide and alkylene oxide from the reactor headspace is circulated and constantly brought into close contact with the liquid and the catalyst. The very high mixing intensity in the injection nozzle reduces the laminar layer thickness on the gas/liquid interface and this improves the mass transfer coefficient.

Suitably, the ejector means (b) is capable of breaking down the gaseous stream into gas bubbles and/or irregularly shaped gas voids. The shearing forces exerted on the suspension in the ejector means (b) may preferably be sufficiently high as to break down the gaseous phase into gas bubbles having diameters in the range of from 1 nm to 10 mm, preferably from 30 nm to 3000 μm, more preferably from 30 μm to 300 μm. A single ejector means may discharge the liquid reaction mixture comprising the gas/liquid mixture into the reactor vessel.

Alternatively, a series of ejector means may be arranged around a tubular loop reactor. The ejector means (b) may be situated inside or outside the reactor vessel. In the latter case, the diffuser may project through the walls of the reactor vessel such that it discharges its contents into the reactor vessel, or the entire ejector means may be situated in the headspace of the reactor. Adjacent to the ejector means (b), there may be provided one or more additional inlets to introduce liquid or gaseous reactants, such as alkylene oxides and/or carbon dioxide. The gaseous and/or liquid components of the reaction may be injected into the reactor vessel for instance via static gas distributors, such as spargers, perforated plates, inserted tubes, gas distributor rings porous sintered appliances such as plugs or dome vents, as described by Wolf-Dieter Deckwer in “Bubble Column Reactors”, Wiley and Sons, 1985, p. 10, which may be located immediately upstream or downstream, preferably upstream of the ejector outlet.

In the downflow jet reactor, the ejector means (b) is situated in the upper part of the reactor, whereas the outlet means (c) is situated in the lower part of the reactor, in such way that at least part of the reaction mixture is extracted from the lower part of the reactor and subsequently re-injected into the reactor through the ejector jet nozzle to create a gas-liquid mixture and to circulate the reaction mixture through the reactor. Thus, the downflow jet reactor is set up in order to make use of a downward flow regime, i.e. the ejector means (b) is situated in the upper part of the reactor, and the outlet means (c) in the lower part of the reactor.

The gas-liquid mixture formed in the ejector means (b) is injected into the reaction medium, preferably below the surface of the reaction medium. Therefore, preferably, the outlet of the ejector means of the downflow jet reactor is located below the surface of a liquid reaction medium present in the reactor. This induces a turbulent flow that increases the interfacial surface area throughout the reactor. This flow then directs the gas-liquid mixture towards the outlet of the reactor, and effects an even distribution of the gas bubbles in the reactor and avoids hotspots through faster heat dissipation that could otherwise lead to an increased generation of by-products. The diffuser or momentum transfer tube of the ejector means is thus preferably constructed in such a way that the gas-liquid mixture that enters the diffuser is impinged on the diffuser walls. This results in the transfer of a part of the kinetic energy of the gas-liquid mixture to the wall surface, resulting in a reduced gas bubble size and again an increase of the gas-liquid surface area.

In ejector reactors the circulation pump is subjected to unusually high damage and wear as compared to what could be expected under the flow and pressure conditions if the reactor was operated for prolonged time periods as typically applied for industrial processes. Furthermore, particles in the circulation conduit due to wear and abrasion of the pump impeller and/or pump housing may damage the ejector, and generally will lead to a contamination and fouling of the reaction conduits. Without wishing to be bound to any particular theory, it is believed that the abrasion of the pump impellers and/or housing is due to gas bubbles that are entrained with the liquid reaction medium. When these gas bubbles reach the circulation pump, they implode immediately when the stream of liquid reaction medium enters the pump impeller chamber where it is decelerated and compressed. Cavitation as a result of such implosions is generally known to induce serious strain and wear on the impeller and pump housing material due to the shock waves that are created at high velocities.

The mixing by the ejector means (b) can produce gas bubbles of such a size so that the interfacial area between gas and liquid is significantly increased compared to bubble cells, and therefore leads to a more effective contact, as described in Ullmann's Encyclopedia of Industrial Chemistry, Vol. B4, 1992, pages 277 and 280.

The actual velocity of the gas/fluid mixture upon entering the reactor will depend on the distance between the ejector means (b) and the reactor, as well as the pressure and the flow of the motivation fluid. The ejector means (b) may advantageously be arranged so that the resulting stream of liquid/gas mixture enters the reactor vessel in a direction parallel or tangential to the longitudinal axis of the reactor. The introduction of the gas/liquid mixture at a relatively high velocity into the comparatively restricted space of the diffuser ensures that gas/liquid mixing is continued and maximized, and that the gas/liquid mixture moves towards the outlet at a rate of 10 m/sec or greater. If the ejector means (b) directs the stream also tangentially to the reactor main axis, the stream will have a large rotational component of velocity, the flow being in a generally helically downward direction. Rotational circulation flow of fluid in the column caused by tangential introduction can lead to the formation of a vortex in the centre of the reactor column immediately below the ejector. The pressure drop of the liquid medium over the ejector means (b) is typically in the range of from 1 to 40 bar, preferably 2 to 30 bar, more preferably 3 to 7 bar, most preferably 3 to 4 bar. Preferably, the ratio of the volume of gas to the volume of liquid passing through the ejector nozzle is in the range of 0.5:1 to 10:1, more preferably 1:1 to 5:1, most preferably 1:1 to 2.5:1, determined at the desired reaction temperature and pressure.

The kinetic energy dissipation rate in the ejector-mixing nozzle is suitably at least 0.5 kW/m³ relative to the total volume of liquid present in the system, and preferably in the range of from 0.5 to 25 kW/m³, more preferably in the range of from 0.5 to 10 kW/m³, and yet more preferably in the range of from 0.5 to 5 kW/m³, and most preferably in the range of from 0.5 to 2.5 kW/m³ relative to the total volume of liquid present in the system.

The reaction liquid may be withdrawn from the lower half of the reactor, preferably from the bottom part of the reactor vessel, and at least in part recycled to the ejector through an external conduit having a first end in communication with an outlet for reaction liquid in the reactor vessel and a second end in communication with an inlet of the ejector. From this outlet, at least part of the reaction mixture may be extracted from the lower part, preferably the bottom, of the reactor and subsequently re-injected into the reactor through the ejector means (b) to create a gas-liquid mixture and to circulate the reaction mixture through the reactor.

The gas-liquid mixture formed in the ejector means (b) is injected into the reaction medium in a downward direction, preferably below the surface of the reaction medium, as this induces a turbulent flow that increases the interfacial surface area throughout the reactor. The downward flow then directs the gas-liquid mixture towards the outlet of the reactor with very high velocity, and effects an even distribution of the gas bubbles in the reactor and avoids hotspots through faster heat dissipation that could otherwise lead to an increased generation of by-products.

The reactor contents are preferably circulated by the means of a conduit tube connecting an outlet in a down-flow direction of the liquid jet, i.e. by a conduit tube that connects an outlet through which the liquid phase that carries the components exits the reaction column to the ejector means (b) for re-injection. This liquid circulation conduit (e) further comprises a circulation pump (f). The circulation pump (f) can be any pump suitable for transporting the large liquid flow. This implies that the pump has to be able to handle the high recycling rate that might be required in order to allow the recycle stream to transfer the heat of reaction to the heat exchanger, as well as the high pressure due to the dissolved gas. Cavitation usually occurs in a fluid flow system when the local static pressure is below the vapor pressure of the gaseous components, hence carbon dioxide and alkylene oxide. However, due to the presence of gas bubbles in the feed stream that enters the pump, the conditions for the cavitation are always present when the liquid/gas-mixture is accelerated in the pump, rendering the problem more severe. Then the gas/vapor bubbles collapse or implode when the fluid velocity is decreased and pressure increased in the pump. This implosion of cavitations is known to cause damage and to trigger side reactions, and leads to increased wear and tear and damage in the pumps, and hence may reduce the lifetime of the components dramatically. Generally, cavitation can be further reduced by reducing the speed of the pump's impeller in centrifugal pumps as commonly applied in industrial scale processes. However, this would require large pumps with huge impeller volumes that can operate at low speed, which is highly undesirable in an industrial scale reactor for the subject process due to the high fluid throughput required for cooling and mixing of the gaseous and liquid components. Alternatively, specifically engineered pumps may be employed that apply a principle different from centrifugal pumps and hence do not suffer from the cavitations, as for instance described by P. Cramers and C. Selinger in an article in Pharm. Chem., June 2002. However, such special pumps are more costly and usually consume more energy, and also require the handling of complex and non-standard technology. As the pump will be exposed to a gas/liquid mixture, cavitation will occur almost immediately upon acceleration and deceleration of the reaction medium upon entry into the pump.

Therefore, in the reactor used in the present invention, by having the gas/liquid mixture impinge on the deflection means (d) situated in between the ejector means and the outlet means in the direction of the flow path of the gas-liquid mixture generated by the ejector means, the presence of gas bubbles in the reaction medium that enters the pump is reduced or completely avoided.

The deflection means (d) is preferably shaped in such way as to deflect the gas/liquid stream away from the outlet. The gas/liquid mixture is deflected on the surface of the deflection means, whereby part of the kinetic energy is transmitted to the deflection means.

As a result, the liquid reaction medium is slowed down, and the gas bubbles present in the liquid medium are deflected away from the outlet. Hence, the gas bubbles will have a velocity that allows the bubbles to coalesce, and to move towards the headspace of the reactor due to their lower density, and not enter the outlet. This area wherein the velocity of the ejected gas/liquid phase is calmed down, is further described herein as calming zone. Accordingly, preferably there is also a calming zone intermediate the ejector means (b) and the outlet, in which calming zone the gas bubbles are allowed to move upward and escape, or to move towards the centre in the case of a vortex when the ejection mode results in a vortex formation. This calming zone and the deflection means (d) may be combined with the outlet design, for instance forming an area wherein the inner walls of the reactor vessel bulge outwardly with an increased diameter, thereby giving the reaction mixture larger space and hence a lower downward velocity prior to passing through the outlet, and/or by baffles or similar structures that slow down the velocity of the reaction mixture.

Dimensions, shape and material of the deflection means (d) also depend largely on the conditions employed. Suitable materials for the deflection means (d) may be chosen in dependence on the velocity, pressure and chemical and physical properties of the reaction medium and its components. The deflection means (d) may have any shape suitable for the above-described deflection and/or calming of the gas/liquid stream, for instance a plate-like, conical, frustroconical or bowl-like shape. Preferably, the deflection means (d) is executed as a conical or bowl-like structure with a diameter larger than the outlet. Yet more preferably, the deflection means has a conical or bowl-like structure and is placed perpendicular to the flow direction such that the outer edges of the deflection means are placed closer to the ejector than the centre of the deflection means.

Preferably the downflow jet reactor used in the process of the invention has a recycle loop and is thus a downflow jet loop reactor.

Further, preferably, the downflow jet reactor used in step (i) of the present process is provided with a device to remove inert gas from the reactor. Inert gasses, like nitrogen, that may be dissolved in the 1,2-alkylene oxide may build up in the reactor, if only liquid is taken out. Therefore, it is preferred to bleed any inert gas phase from the reactor.

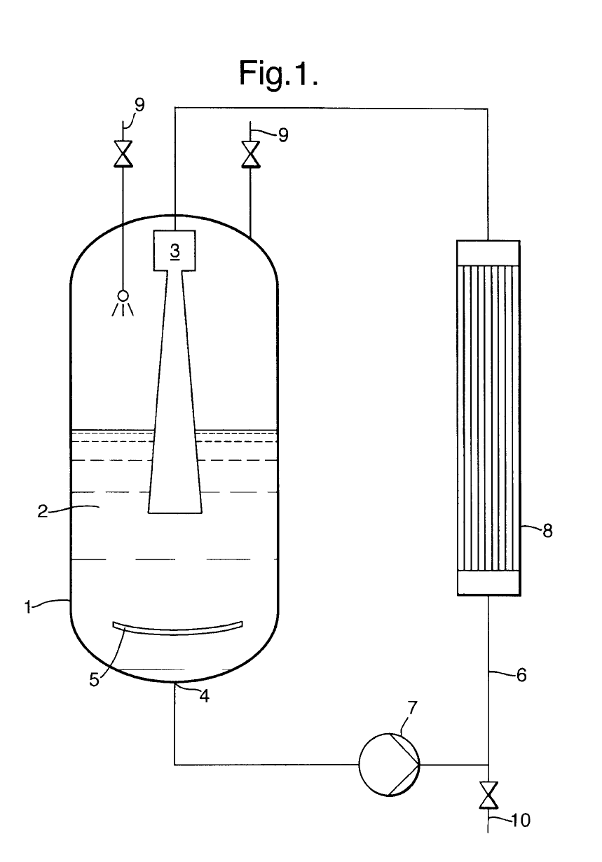

FIG. 1 depicts a preferred embodiment of the downflow jet reactor for contacting gas and liquid which may be used in the process of the present invention, comprising: a reactor vessel (1) comprising a liquid reaction medium (2); an ejector means (3) suitable for mixing the gas and the liquid and ejecting the gas/liquid mixture obtained into the reactor vessel; an outlet means (4), a deflection means (5) positioned in the direct flow path between the ejector and the outlet means in flow direction of the gas/liquid stream; a liquid circulation conduit (6) connecting the outlet means and the ejector means to effect circulation of the reaction medium (2) from the outlet (4) into the ejector means (3), a circulation pump (7) situated in the circulation conduit, a heat exchanger (8) situated in the circulation conduit, one or more additional fluid and/or gas inlets (9) and a liquid outlet (10) situated in the circulation conduit to allow at least part of the reaction medium to be diverted.

The carbon dioxide and/or 1,2-alkylene oxide may be fully or partially dosed into the recycle loop of a downflow jet loop reactor, for example into the above-mentioned circulation conduit (6) as shown in FIG. 1. Further, a catalyst make-up stream may be dosed directly into the downflow jet reactor, for example into the above-mentioned reactor vessel (1) as shown in FIG. 1, and/or into the recycle loop of a downflow jet loop reactor, for example into the above-mentioned circulation conduit (6) as shown in FIG. 1.

The reaction in step (i) of the process of the invention is believed to mainly occur in the gas/liquid mixture formed by the liquid reaction medium and the gaseous phase.

The liquid reaction medium may conveniently be composed of 1,2-alkylene diol and liquefied alkylene oxide when the reaction is started up, however during operation of the subject process, it is preferably composed of the alkylene carbonate that is formed in the reaction.

In the above process, the presence of alcohol is not required in step (i), while preferably alcohol is absent. Accordingly, although the subject process can tolerate in step (i) the presence of water, suitably, in step (i), less than an equimolar amount of water is present calculated on the basis of the alkylene oxide. Preferably, step (i) is performed in the presence of less than 10% by weight of total amount of water and/or alcohol, calculated on the total amount of reactants, yet more preferably less than 5% by weight, still more preferably less than 3% by weight, again more preferably less than 1.5% by weight, even more preferably less than 1% by weight, and most preferably less than 0.5% by weight of water to avoid side reactions. Again, preferably the combined process feeds of step (i) contain from 0 to 5% by weight of total amount of water, more preferably, the feeds contain from 0 to 3% by weight of water, again more preferably less than 2% by weight, yet more preferably less than 1% by weight, again more preferably less than 0.5% by weight, and most preferably, less than 0.1% by weight of total amount of water, calculated on the basis of the amount of alkylene oxide present. As a result, in the second reaction stage, alcohol can be dosed in such amounts that the 1,2-alkylene diol can be obtained in a water/alcohol-free solution, which does not require distillation and separation of the 1,2-alkylene diol, which requires an unnecessarily high amount of energy. Further, the selectivity of the subject process is increased compared to processes wherein water is present in the presence of both alkylene oxide and 1,2-alkylene diol. The subject process further has the advantage of being suitable for a continuous operation on an industrial scale without involving cumbersome catalyst refining steps and without handling of solids.

Preferably, the reaction mixture obtained in step (i) is at least in part recycled to step (i), including any unreacted alkylene oxide, the formed alkylene carbonate and any alkylene glycol together with the catalyst. Accordingly, the reactor of step (i) is advantageously constructed as a loop reactor.

In particular, it was found that recycling of the carbonation catalyst from the transesterification stage in step (ii) to the carbonation stage in step (i) can be advantageously done by dissolving the catalyst in a 1,2-alkylene diol without increased formation of side products, provided that water and/or alcohol is not present in an excessive amount in the alkylene oxide in the first reaction step, while at the same time increasing the heat capacity of the reaction mixture and stabilizing the catalysts is applied. Its presence also allows simple start-up procedures and control over the reactor heat through its high boiling point and low vapor pressure, and no undue increase of pressure due to decomposition, as in the case of alkylene carbonate.

In a loop reactor, at least part of the reaction mixture including alkylene carbonate and any 1,2-alkylene diol is recycled back into the reactor as motive liquid. This allows use of the recycled liquid medium as motive liquid to form the gas/liquid mixture, and permits to operate a relatively small reactor although achieving high throughput, while also providing for easy control of the heat of the reaction through an external heat exchanger.

In the present process, it was found that the reaction rate of step (i) and hence the conversion can be controlled in a simple way through the recycle ratio of added alkylene oxide and carbon dioxide to the recycled motive liquid. Therefore the reactor set-up for step (i) preferably comprises a reactor vessel having the ejector means in the uppermost part of the reactor vessel, an outlet in the lower part of the reactor vessel, and a recycle line that connects the outlet to a circulation pump and further to the inlet for motive fluid of the ejector means. The recycle line further preferably comprises a heat exchanger to remove at least part of the heat of reaction. In step (i) of the subject process, preferably a recycle ratio in the range of from 1 to 1000 by volume of the recycled medium per unit of time to alkylene carbonate formed per unit of time is maintained to control the heat of reaction. More preferably, a ratio of from 2 to 800, and yet more preferably a ratio of from 6 to 400, again more preferably a ratio from 7 to 350 by volume of the recycled medium per unit of time to alkylene carbonate formed per unit of time is maintained, which allows control of the reactor temperature and turnover rate.

The circulation pump for the recycling of the reaction mixture may be any pump suitable for the recycling rate and the flow required. In particular, the pump has to be able to accommodate for high flow that might be required for a suitable heat transfer to the heat exchanger. The heat exchanger may be any suitable heat exchanger. Preferably, the heat exchanger is a shell and tube heat exchanger for efficient heat removal, as the reaction of alkylene oxide and carbon dioxide is highly exothermic, and the alkylene carbonate formed becomes unstable at increased temperatures.

The circulation pump increases the pressure from the pressure at the outlet of the loop reactor in order to avoid cavitation in the pump, and may maintain a pressure differential over the two reaction steps (i) and (ii) that allows the subject process to be conducted without additional means to provide pressure. Preferably, a single circulation pump is employed to recycle the reaction mixture as well as to provide for the pressure increase to step (ii), and to transport part of the reaction mixture to the reactor of step (ii).The circulation pump is preferably situated below the outlet in order to increase the hydrostatic pressure in order to reduce cavitations in the pump. More preferably, the circulation pump is situated at least 1 m, more preferably 3 m, and most preferably 5 m below the reactor vessel. Conveniently, the loop reactor of step (i) is a reactor as described in U.S. Pat. No. 5,159,092. Such a reactor set-up achieves a much higher mixing than cascades of bubble-cell reactors, in particular if the injection is performed downwards, whereas in bubble flow reactors, the flow regime and the mixing is mainly governed by gravitational forces and density of the gas bubbles versus the density of the motive liquid.

Suitable alkylene oxides include lower alkylene oxides, such as ethylene oxide, propylene oxide and butylene oxide, as well as heavier and functionalized alkylene oxides, such as styrene oxide. Preferably, the alkylene oxide has from 2 to 15 carbon atoms, yet more preferably, the alkylene oxide has from 2 to 8 carbon atoms, again more preferably from 2 to 4 carbon atoms. Most preferably, the 1,2-alkylene oxide is 1,2-ethylene oxide, 1,2-propylene oxide or a mixture thereof.

Suitable carbonation catalysts for step (i) are homogeneous catalysts, i.e. catalysts that dissolve in the reaction medium. Although heterogeneous catalysts have also been described as suitable, such heterogeneous catalysts have the disadvantage that due to the large fluid streams present in a jet loop reactor, the catalyst particles tend to erode themselves as well as the nozzle(s) of the ejector means rather quickly. Suitable homogeneous carbonation catalysts for step (i) include alkali or alkaline earth metal halides, tertiary and quaternary ammonium, phosphonium and sulfonium salts, tertiary phosphines and nitrogen bases. Within the quaternary phosphonium halide family, the suitability for use as catalyst for the subject process has now been found to depend on the halide counter-ion, as well as on the structure of the phosphonium moiety. Halides are ions of F, Cl, Br, I and At. Of these, astatine-containing compounds are not used due to the radioactivity of the element and its low availability. Equally, quaternary phosphonium fluorides are usually not used due to the low environmental acceptance of fluorine containing side products. Accordingly, the carbonation catalyst preferably is a tetraalkyl phosphonium halide, yet more preferably a tetraalkyl phosphonium bromide. Such tetraalkyl phosphonium bromide may have the formula R¹R²R³R⁴PBr (I). Tetraalkyl within the sense of the present invention means that the alkyl substituents R¹ to R⁴ are covalently bonded to the phosphorus atom. Preferably, alkyl substituent means a saturated hydrocarbon radical having from 1 to 10 carbon atoms, more preferably from 1 to 6, again more preferably from 2 to 4 carbon atoms, and most preferably 4 carbon atoms. Accordingly, the preferred alkyl substituents R¹ to R⁴ are preferably selected from the group consisting of methyl, ethyl, n-propyl, isopropyl, n-butyl, isobutyl and tertiary butyl, the most preferred alkyl substituent being n-butyl. Within the tetraalkyl phosphonium bromides, symmetrically substituted tetraalkyl phosphonium bromides, i.e. those wherein the four alkyl substituents are identical alkyl radicals, were found to be more stable than asymmetrically substituted tetraalkyl phosphonium bromides at similar activity levels. Accordingly, a tetraalkyl phosphonium bromide catalyst of the formula R¹R²R³R⁴PBr (I), wherein R¹, R², R³ and R⁴ in formula (I) represent identical alkyl groups is preferably used in the subject process. An additional advantage for the use of a symmetrically substituted tetraalkyl phosphonium bromide catalyst resides in the fact that simpler decomposition product mixtures are formed than by using asymmetrically substituted phosphonium catalysts. This allows a more efficient purification of the desired end products. Therefore, in step (i), the subject process preferably employs tetra-n-butyl phosphonium bromide as the catalyst. This catalyst has the further advantage that it dissolves readily in the formed alkylene carbonate and any 1,2 alkylene diol, and to some extent in the alkylene oxide. The amount of catalyst may conveniently be expressed in mole catalyst per mole alkylene oxide.

Step (i) may be conducted at varying catalyst concentrations. The determination of a particular effective concentration largely depends on the process parameters; such as for instance residence time of the process feeds in the reactor, type of feed, temperature, and pressure. The amount of catalyst may conveniently be expressed in mole catalyst per mole alkylene oxide. Preferably due to a lower amount of by-products, the subject process is performed in the presence of at least 0.0001 mole catalyst per mole alkylene oxide. Yet more preferably, the subject process is performed using a ratio in the range of from 0.0001 to 0.1 mole catalyst per mole alkylene oxide, more preferably of from 0.001 to 0.05, and most preferably of from 0.003 to 0.03 mole catalyst per mole alkylene oxide.

The insertion of carbon dioxide into the oxirane moiety of alkylene oxides is a reversible reaction, i.e. alkylene oxide may also be formed back from alkylene carbonate under release of carbon dioxide. In order to shift the equilibrium towards the desired alkylene carbonates, the reaction is preferably performed under increased pressure. Besides providing for the desired surplus of carbon dioxide, operation at increased pressure also permits to conduct the reaction essentially in the liquid phase, as particularly ethylene oxide and propylene oxide will largely remain liquid under the process conditions. Step (i) is thus preferably conducted at a total pressure in the range of from 0.5 to 20 MPa (i.e. 5 to 200 bar), with a partial carbon dioxide pressure preferably being in the range of from 0.5 to 7 MPa, more preferably in the range of from 0.7 to 5 MPa, and most preferably in the range of from 1 to 2 MPa. More preferably, step (i) is performed at a pressure in the range of from 1 to 15 MPa, yet more preferably in the range of from 1.5 to 10 MPa, and most preferably in the range of from 1.8 to 5 MPa.

Conveniently, in step (i), the reactor content is maintained at a temperature in the range of from 150° C. to 190° C., yet more preferably in the range of from 160° C. to 180° C.

The subject process is preferably performed in such way that in step (i) the molar ratio of carbon dioxide to 1,2-alkylene oxide is between 0.6 and 0.99, more preferably between 0.8 and 0.98.

In step (ii) of the process of the invention, at least part of the reaction mixture obtained in step (i) is contacted with an alkanol to obtain 1,2-alkylene diol and dialkylcarbonate. Step (ii) can be carried out with any alkanol known to be suitable for this purpose. The alkanol can be a mono- or poly-alkanol, but is preferably a mono-alkanol. More preferably step (ii) is carried out with a mono-alkanol having from 1 to 4 carbon atoms, such as methanol, ethanol, isopropanol, n-propanol, n-butanol, sec-butanol or tert-butanol. Preferably the alkanol is methanol, ethanol or isopropanol, such that mono-alkylene glycol and respectively dimethylcarbonate, diethylcarbonate or di-isopropyl-carbonate can be obtained. The most preferred alkanols are methanol and ethanol.

The alkanol may be added in step (ii) in any suitable amount. However, it preferably is only added in an amount equal to or only slightly above the molar amount of alkylene carbonate, and any alkylene oxide present in the reactor. This results in a reaction mixture wherein the 1,2-alkylene diol is highly concentrated, which in turn makes the separation and purification of the 1,2-alkylene diol highly energy-efficient.

Although the catalyst employed in step (i) may also catalyze the transesterification reaction of step (ii), the latter reaction is preferably conducted in the presence of a heterogeneous transesterification catalyst. Suitable catalysts include basic or acidic solids, such as ion exchange resins, or mineral materials that comprise metal hydroxide structures, such as alumina or titanium. Preferably, step (ii) is performed in the presence of a heterogeneous transesterification catalyst. Suitable heterogeneous transesterification catalysts in particular include ion exchange resins with tertiary amine, quaternary ammonium, sulfonic acid and carboxylic acid functional groups; and alkali and alkaline earth silicates impregnated into silica and ammonium exchanged zeolites. Further suitable catalysts include alkali metal compounds, in particular alkali metal hydroxides or alcoholates, thallium compounds, nitrogen-containing bases such as trialkyl amines, phosphines, stibines, arsenines, sulfur or selenium compounds and tin, titanium or zirconium salts.

Step (ii) may be performed in any suitable reactor, such as a trickle bed reactor, preferably a multi-tubular reactor with co-continuous liquid phase as well as gas phase, and more preferably with co-current downward flow, yet more preferably followed by gas-liquid phase separation.

If the second reaction step (ii) requires heat input, this may be provided by heating through external and/or internal heat exchangers filled with steam or any other suitable heat transfer medium that can be circulated continuously throughout plant operations at the required temperatures. The reaction of propylene carbonate with an alkanol is slightly endothermic and the reaction of ethylene carbonate with an alkanol is slightly exothermic.

Step (ii) is preferably conducted at a total pressure in the range of from 1.5 to 25 MPa (i.e. 15 to 250 bar). More preferably, step (ii) is performed at a pressure in the range of from 1.0 to 15 MPa, yet more preferably in the range of from 1.5 to 10 MPa, and most preferably in the range of from 2 to 5 MPa. Preferably, in the subject process, step (ii) is conducted at a pressure of at least 0.3 MPa (3 bar) higher pressure than step (i), yet more preferably at least 0.5 MPa higher. This higher pressure also appears to beneficially reduce the formation of side products. Preferably, step (ii) is conducted at a lower temperature than step (i), which results in a higher selectivity, as less side products are formed. Most preferably, step (i) is conducted at a pressure of from 1.5 to 2.5 MPa and a temperature in the range of from 150° C. to 190° C., and step (b) at 2 to 3 MPa and in a temperature range of from 100° C. to 140° C.

Alkanol is preferably added in step (ii) in a larger than equimolar amount required to convert the alkylene carbonate and optional alkylene oxide present, thereby permitting high conversion.

The mixture obtained from step (ii) may contain 1,2-alkylene diol, dialkylcarbonate, the catalyst of step (i) and any side products of the reactions of steps (i) and (ii). Preferably, in the subject process, the reaction mixture obtained in step (i) is separated prior to step (ii) into a gaseous stream comprising carbon dioxide and a liquid stream comprising the carbonation catalyst and any 1,2-alkylene diol, wherein the gaseous stream is returned to step (i).

Preferably, the reactor of step (i) is equipped with a gas circulation overhead washer. The carbon dioxide that is recycled to the reaction of step (i) may then conveniently be introduced in counter flow to any gas passing into the gas-overhead, and thereby return any alkylene oxide to step (i).

In step (iii) of the process of the present invention, the 1,2-alkylene diol and the dialkylcarbonate are recovered from the reaction mixture obtained in step (ii).

Preferably, said step (iii) comprises the steps of (iii)(a) separating the mixture obtained in step (ii) into a fraction comprising part of the 1,2-alkylene diol and a second fraction comprising the carbonation catalyst dissolved in part of the 1,2-alkylene diol; and (iii)(b) recycling the second fraction obtained in step (iii)(a) to step (i).

The separation in step (iii)(a) may be done by distillation, for instance a flash distillation, or by any other suitable method of separation. Preferably, not all 1,2-alkylene diol is separated from the reaction mixture obtained in step (ii) so the catalyst remains dissolved for recycling back in step (iii)(b) into the carbonation reaction of step (i).

The separation in step (iii)(a) may be achieved in several ways. First of all, dialkylcarbonate may be separated by distillation into an overhead stream from a bottoms stream comprising 1,2-alkylene diol and carbonation catalyst. Said bottoms stream is then separated into a fraction comprising part of 1,2-alkylene diol and a second fraction comprising carbonation catalyst dissolved in part of 1,2-alkylene diol, said second fraction to be recycled to step (i). A second way is that dialkylcarbonate and part of 1,2-alkylene diol are separated by distillation into an overhead stream from a bottoms stream comprising carbonation catalyst dissolved in part of 1,2-alkylene diol, said bottoms stream to be recycled to step (i). Subsequently, the overhead stream comprising dialkylcarbonate and part of 1,2-alkylene diol is separated into an overhead stream comprising dialkylcarbonate and a bottoms stream comprising 1,2-alkylene diol.

Any crude catalyst recycle stream contains the catalyst and possible by-products, which can conveniently be separated as a bleed stream from the catalyst stream.

Generally, there are problems associated with using homogeneous catalysts in industrial scale processes. The problems include loss of activity during recycling and, the requirement of the disposal of a large amount of inactive spent catalyst, in particular with highly active catalysts such as tetraalkyl ammonium halides, tetraalkyl phosphonium halides and guanidinium halides. As a result, catalyst breakdown products accumulate in the reaction medium, while the reaction proceeds at a slower rate. Also, the catalyst breakdown products in the reaction product stream might necessitate extensive purification of the desired product.

Moreover, the 1,2-alkylene diol obtained in step (ii) appears to stabilize the catalyst, and thus permits operation of step (i) at a higher temperature and hence higher turn over rate without leading to a significant increase of side-products. The 1,2-alkylene diol also increases heat capacity of the reaction mixture of the reactor used in step (i) and thus allows a better heat-dissipation and cooling, and thus may contribute to an increase of the potential load and hence capacity of the reactor of step (i). The catalyst stream is preferably recycled to step (i) by injecting it through the ejector means with the motive liquid.

As another preferred alternative, in step (i) of the process of the present invention the 1,2-alkylene carbonate is prepared by a process comprising
(a) contacting carbon dioxide, a 1,2-alkylene oxide and a carbonation catalyst in a downflow jet reactor to produce a crude reactor effluent containing carbon dioxide, light components, 1,2-alkylene carbonate and catalyst;
(b) separating carbon dioxide and light components from the crude reactor effluent to form a bottoms stream containing 1,2-alkylene carbonate and catalyst;
(c) distilling the bottoms stream formed in step (b) to form a first distillation overhead stream containing 1,2-alkylene carbonate and a first distillation bottoms stream containing catalyst, and recycling at least part of the first distillation bottoms stream to the reactor; and
(d) distilling the first distillation overhead stream to form a second distillation overhead stream and a second distillation bottoms stream containing 1,2-alkylene carbonate, and recycling at least part of the second distillation overhead stream to the reactor.

In accordance with the present description, light components are compounds, other than carbon dioxide, which have a boiling point which is lower than that of 1,2-alkylene glycols and 1,2-alkylene carbonates, more specifically 185° C. or lower, and most specifically 180° C. or lower. Examples of such light components in the crude effluent from the carbonation reactor may be unreacted 1,2-alkylene oxide and any light contaminants formed during the carbonation reaction, such as acetone, propionaldehyde, allyl alcohol and acetaldehyde.

What is claimed is:

1. A process for the preparation of a 1,2-alkylene diol and a dialkylcarbonate, comprising:
(i) contacting a 1,2-alkylene oxide with carbon dioxide in the presence of a carbonation catalyst in a downflow jet reactor to obtain a reaction mixture containing a 1,2-alkylene carbonate,
wherein the downflow jet reactor is a reactor comprising a reactor vessel, an ejector means suitable for mixing the gas and the liquid and ejecting the gas/liquid mixture obtained into the reactor vessel, and an outlet means, wherein the ejector means is situated in the upper part of the reactor vessel and the outlet means is situated in the lower part of the reactor vessel, which reactor is operated in a downflow fashion, and
wherein the downflow jet reactor further comprises a deflection means situated in between the ejector means and the outlet means in the direction of the flow path of the gas/liquid mixture generated by the ejector means;
(ii) contacting at least part of the reaction mixture obtained in step (i) with an alkanol to obtain a reaction mixture containing a 1,2-alkylene diol and a dialkylcarbonate; and
(iii) recovering the 1,2-alkylene diol and the dialkylcarbonate from the reaction mixture obtained in step (ii).

2. A process as claimed in claim 1, wherein step (iii) comprises:
(iii)(a) separating the mixture obtained in step (ii) into a fraction comprising part of the 1,2-alkylene diol and a second fraction comprising the carbonation catalyst dissolved in part of the 1,2-alkylene diol; and
(iii)(b) recycling the second fraction obtained in step (iii)(a) to step (i).

3. A process as claimed in claim 1, wherein the molar ratio of carbon dioxide to the 1,2-alkylene oxide in step (i) is between 0.6 and 0.99.

4. A process as claimed in claim 1, wherein the carbonation catalyst comprises a tetra-alkylphosphonium bromide.

5. A process as claimed in claim 1, wherein the deflection means has a conical or bowl-like structure and is placed perpendicular to the flow direction such that the outer edges of the deflection means are placed closer to the ejector than the centre of the deflection means.

6. A process as claimed in claim 1, wherein the outlet of the ejector means of the downflow jet reactor is located below the surface of a liquid reaction medium present in the reactor.

7. A process as claimed in claim 1, wherein the downflow jet reactor is provided with a device to remove inert gas from the reactor.

8. A process as claimed in claim 1, wherein step (ii) is conducted in the presence of a heterogeneous transesterification catalyst.

9. A process as claimed in claim 1, wherein the 1,2-alkylene oxide is selected from the group consisting of 1,2-ethylene oxide, 1,2-propylene oxide and mixtures thereof.
